(19) United States
(12) Patent Application Publication
(10) Pub. No.: US 2015/0374943 A1
(43) Pub. Date: Dec. 31, 2015

(54) FLEXIBLE CUSHION SYSTEM FOR OXYGEN MASK

(71) Applicant: Alex Alexani, Glendale, CA (US)

(72) Inventor: Alex Alexani, Glendale, CA (US)

(21) Appl. No.: 14/320,186

(22) Filed: Jun. 30, 2014

(51) Int. Cl. A61M 16/06; A61M 16/10

(52) U.S. Cl. CPC A61M 16/0622; A61M 16/0694; A61M 16/0688; A61M 16/1005; A61M 2240/00

(57) ABSTRACT

A flexible cushion system for oxygen mask comprises an oxygen mask for providing oxygen gas from a gas source to a user to breathe in the gas through the user's nose. A cushion is provided for connecting to the mask for protecting and comforting the user's face. In one embodiment, the cushion comprises a nasal bridge section for connecting to a mask section that places on wearer's nose, a cheek section for connecting to a mask section that places on wearer's cheek, and a chin section for connecting to a mask section that places on wearer's chin. In one embodiment the cheek section and the chin section are separate pieces. Preferably, each of the nasal bridge section, the cheek section and the chin section are separable from the mask. In another embodiment the cushion is in one piece. Preferably, the cushion thickness can be reduced from an initial thickness by one or more perforations along its original surface.

### BACKGROUND OF THE INVENTION

### Field of the Invention

The present invention relates to a cushioning system for use on Oxygen masks used to transfer oxygen from a storage to the user who breathes the gas through the user's nose.

An oxygen mask is often used by patients in hospitals and at home, as method to transfer breathing oxygen gas from a storage tank to the lungs. Oxygen masks may cover the nose and mouth (oral nasal mask) or the entire face (full-face mask). They may be made of plastic, silicone, rubber or other such material.

Conventional Oxygen masks are light in weight and attached using an elasticated headband or ear loops, they are transparent allowing the face visible for patient assessment by healthcare provider and reducing a sensation of claustrophobia experienced by some patients when wearing an oxygen mask. The vast majority of patients having an operation will at some stage wear an oxygen mask.

A disadvantage of the conventional masks is the relative stiffness and rigidity of the material of which the masks are made compared to the soft and tender skin of the human face. Conventional masks include walls which are typically made of silicon or other such flexible material and the masks are normally secured to the wearer's face by straps around the user's head. The resulting friction and pressure by the mask against user's face can cause irritation, swelling or worse.

Due to excessive pressure on the user's face by the face contacting portion of the mask, user may experience discomfort and possibly skin irritation. This occurs because the face contacting portion of the mask has to distort beyond its normal range of elasticity to conform to certain facial contours which requires the application of excessive forces. In some cases these excessive pressures and forces may cause the face to distort to conform with the face contacting portion to increase wearer discomfort, facial soreness and ulceration.

There is therefore a need for an oxygen mask cushion system that addresses the above noted deficiencies of the conventional masks.

### SUMMARY OF THE DISCLOSURE

A flexible cushion system for oxygen mask comprises an oxygen mask for providing oxygen gas from a gas source to a user to breathe in the gas through the user's nose. A cushion connects to the mask for protecting and comforting the user's face and to help prevent facial soreness and ulceration. In one embodiment, the inventive cushion system comprises several separate pieces that can be individually attached or separated from the mask. This embodiment allows user to adapt and adjust the mask according to user's requirements and preferences. According to this embodiment the cushion includes a nasal bridge section which attaches to a mask section that places on user's nose, a two-piece cheek section corresponding to mask areas that place on user's cheek on each side of the mask, and a chin section for attaching to a mask area that places on user's chin.

In another embodiment the inventive oxygen mask cushion is in one piece and placed inside the mask and extending to the outside. Advantageously, the width of the cushion is adjustable to accommodate user's requirements and preferences, including various face sizes corresponding to those of children, adults and other variants in face sizes and shapes. Adjustability of the cushion width is preferably accomplished through inclusion of perforated lines along the width of the cushion that allows for easy reduction of the cushion width.

In yet another embodiment, the inventive oxygen mask cushion is in multiple pieces with the width of each piece being adjustable.

Advantageously, the inventive oxygen mask cushion system protects user's nose skin from Necrosis which is caused by excessive pressure and factors external to the cell or tissue, such as infection, toxins, or trauma that result in the unregulated digestion of cell components.

Advantageously, the inventive oxygen mask cushion system provides more stability than the conventional masks by flexibly and adjustably cushioning the mask on the patient face.

Advantageously, the inventive oxygen mask cushion system is suitable for user's of all ages and various face shapes and sizes including adults, children and infants.

Advantageously, the inventive oxygen mask cushion system is easy to put on and to remove and easy to apply cushion and adjustable on patient skin and mask for example, by way of a one sided taped cushion to be taped on user's skin or on the mask.

Advantageously, the inventive oxygen mask cushion system is disposable and inexpensive and can result in significant cost savings for Medicaid and Medicare as well as private insurance.

Advantageously, the inventive oxygen mask cushion system is simple to assemble and can be used in emergencies as well as short-term, and long-term oxygen therapy cases.

Advantageously, the inventive cushion system can be used on simple oxygen masks, regular aerosol oxygen masks, air entrainment masks (venti-mask), non re breathable (NRB) masks and the like.

Advantageously, the inventive mask cushion system provides for cushion and pad on mask to go around and inside the mask and therefore cover the entire area where there is a contact with skin.

### BRIEF DESCRIPTION OF THE DRAWINGS

FIG. 1 is a perspective view of an oxygen mask to be equipped with the inventive cushion mask.

FIG. 2 is a perspective view of the constituent parts of the inventive oxygen mask cushion system according to one exemplary embodiment of the present invention.

FIG. 3 is a perspective view of the constituent parts of the inventive oxygen mask cushion system according to another exemplary embodiment of the present invention.

### DETAILED DESCRIPTION OF PREFERRED EMBODIMENTS

The various aspects and features of some exemplary embodiments of the present invention will now be discussed with reference to the drawings.

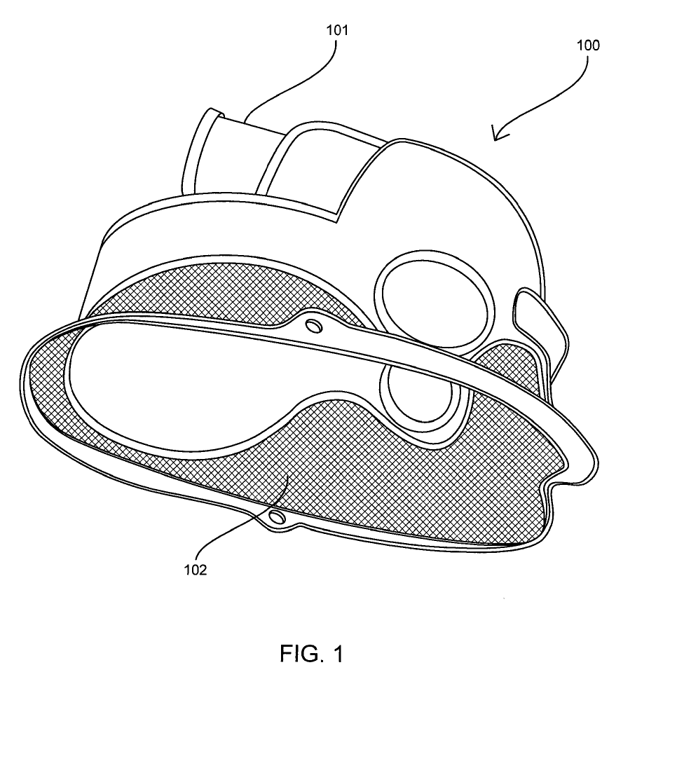

FIG. 1 is a perspective view of an oxygen mask to be equipped with the inventive cushion mask. The mask 100 includes an air inlet port 101 and a periphery 102. When worn by a user (not shown) without the inventive cushion system, the peripheral areas 102 of the mask 100 are in touch with the user's face.

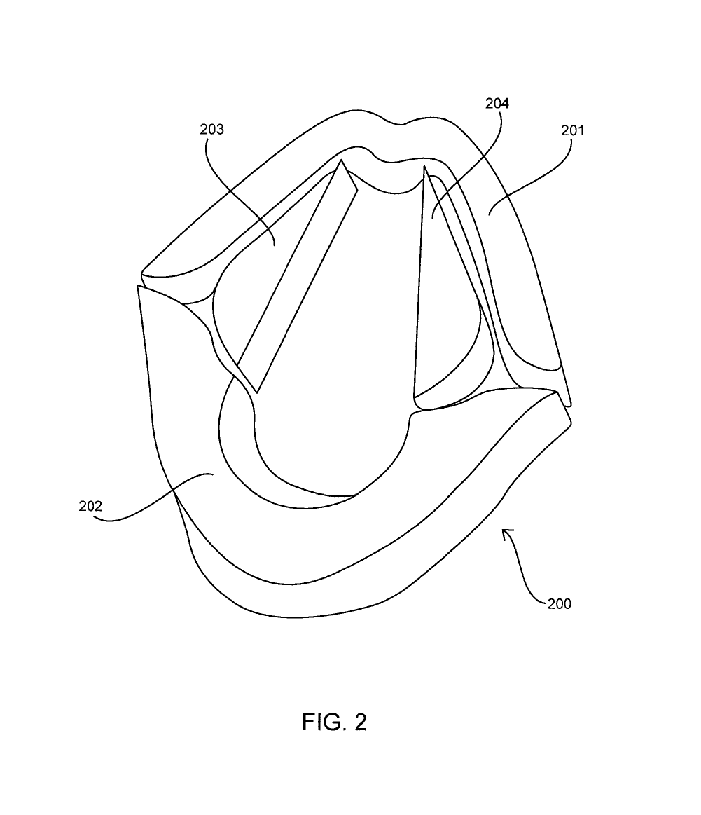

Referring now to the invention in more detail with reference to FIG. 2, the inventive mask cushion system is shown according to an exemplary embodiment. Reference numeral 200 identifies the oxygen mask cushion that would be fitted with an oxygen mask to create the inventive oxygen mask system. The inventive cushion of this embodiment includes multiple cushion pieces that are each attachable and detachable from mask 200. Nose piece 201 fits inside the oxygen mask and would be in touch with user's nasal bridge and its surrounding tissue under user's eyes. Cheek pieces 203 and 204 cover the length of user's nose along each of its sides. Chin piece 202 covers the user's chin and its surrounding areas. Each of the pieces are separately attachable to and detachable from mask 200.

Advantageously, separability of the cushion's individual pieces provides the user with the flexibility to configure a mask cushion according to user's preferences and requirements.

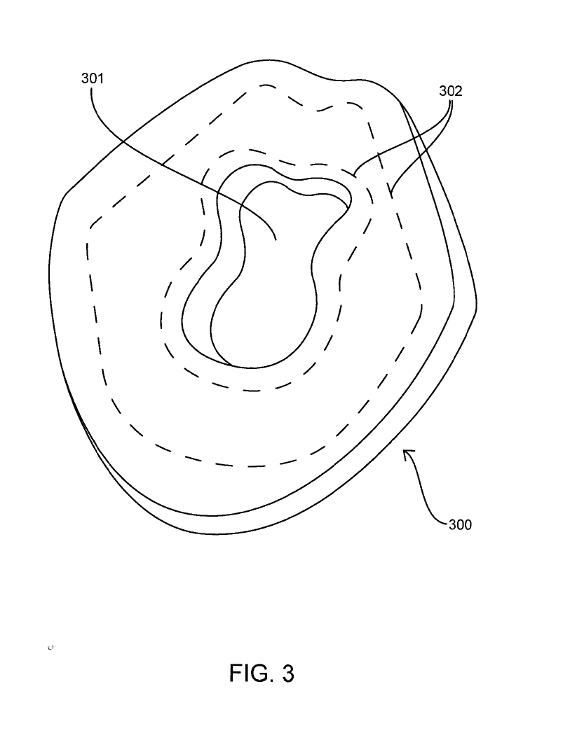

Referring now to the invention in more detail in FIG. 3, the inventive mask cushion system is shown according to another exemplary embodiment. Reference numeral 300 identifies the oxygen mask cushion that would be fitted with an oxygen mask to create the inventive oxygen mask system. In this embodiment, the mask cushion 200 is in once piece and the width of the mask is adjustable. The adjustment in the width of the mask is accomplished by cutting the width along perforated lines 302.

Advantageously, the adjustability of the cushion width reduces skin contact at the same locations, which, in turn, reduces the adverse effects of prolonged contact between user's skin and the cushion in the same areas.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. A flexible cushion system for oxygen mask comprising:
An oxygen mask for providing oxygen gas from a gas source to a user to breathe in the gas through the user's nose;
A cushion for connecting to the mask for protecting and comforting the user's face the cushion comprising:
A nasal bridge section for connecting to a mask section that places on wearer's nose;
cheek sections for connecting to a mask section that places on wearer's cheek; and
A chin section for connecting to a mask section that places on wearer's chin.

2. The mask cushion system of claim 1 wherein each of the nasal bridge section, the cheek section and the chin section are separate pieces.

3. The mask cushion system of claim 2, wherein each of the nasal bridge section, the cheek section and the chin section are separable from the mask.

4. A flexible cushion system for oxygen mask comprising:
An oxygen mask for providing oxygen gas from a gas source to a user to breathe in the gas through the user's nose;
A cushion for connecting to the mask for protecting and comforting the user's face, wherein the cushion is in one piece.

5. The mask cushion system of claim 4, wherein the cushion thickness can be reduced from an initial thickness.

6. The mask cushion system of claim 5, wherein the reduction in cushion thickness is accomplished by one or more perforations along its original surface.
